(19) United States
(12) Patent Application Publication
(10) Pub. No.: US 2004/0058006 A1
(43) Pub. Date: Mar. 25, 2004

(54) High affinity nanoparticles

(75) Inventors: Stephen E. Barry, Oakland, CA (US); David S. Soane, Piedmont, CA (US)

Correspondence Address: JACQUELINE S LARSON, P O BOX 2426, SANTA CLARA, CA, 95055-2426, US

(73) Assignee: Alnis BioSciences, Inc.

(21) Appl. No.: 10/667,635

(22) Filed: Sep. 22, 2003

Related U.S. Application Data
(63) Continuation-in-part of application No. 09/809,340, filed on Mar. 14, 2001, now abandoned.
(63) Continuation-in-part of application No. 10/055,837, filed on Oct. 26, 2001.
Application No. 10/055,837 is a continuation of application No. 09/172,921, filed on Oct. 14, 1998, now abandoned.
(60) Provisional application No. 60/189,625, filed on Mar. 14, 2000.
(60) Provisional application No. 60/061,805, filed on Oct. 14, 1997.
(60) Provisional application No. 60/103,616, filed on Oct. 9, 1998.

(51) Int. Cl. A61K 38/16; A61K 31/70; A61K 39/395; A61K 31/56; A61K 31/555; A61K 9/14

(52) U.S. Cl. 424/489; 514/8; 514/12; 514/44; 514/23; 514/169; 514/185; 514/410; 424/85.1; 424/185.1; 424/130.1

(57) ABSTRACT

High affinity nanoparticles are provided, as well as methods for their synthesis and use. The nanoparticles of the invention comprise high affinity molecules incorporated in a polymeric nanoparticle. The high affinity nanoparticles range in size from about 1 to about 1000 nm. The high affinity molecules of the nanoparticle have moieties that have high affinity for target molecules, resulting in the ability of the high affinity nanoparticle to selectively non-covalently bind to molecular targets. The molecular recognition capability of these particles enables their use in research, diagnostic, therapeutic, and separation applications. The nanoparticles of the invention may be formed by contacting target template molecules with a set of building blocks (which includes the high affinity molecule as one subset of the building block set), which are then polymerized into a network. Removal of the templates yields a polymeric nanoparticle with three-dimensional binding sites that are complementary in shape to at least a portion of the target and including high affinity molecules chemically anchored on the surfaces of the binding sites. The high affinity nanoparticle is then capable of molecular recognition and selective binding to target molecules when presented with the target molecule in a mixture of molecules.

This application is a continuation-in-part of copending U.S. application Ser. No. 09/809,340, filed Mar. 14, 2001, which application claims the benefit of co-pending Provisional patent application Serial No. 60/189,625, filed Mar. 14, 2000. This application is also a continuation-in-part of copending U.S. application Ser. No. 10/055,837, filed Oct. 26, 2001, which is a continuation of application Ser. No. 09/172,921, filed Oct. 14, 1998, which application claims the benefit of U.S. provisional patent application serial No. 60/061,805, filed Oct. 14, 1997, and U.S. provisional patent application serial No. 60/103,616, filed Oct. 9, 1998. The entire disclosures of all of the above applications are incorporated herein by reference.

### TECHNICAL FIELD

This invention relates to the creation of polymer nanoparticles capable of specifically binding to biological molecular targets, and to the manufacture of such polymer nanoparticles.

### BACKGROUND

Molecular recognition events are ubiquitous in life and the cornerstone of the pharmaceutical and biotechnology industries. Some examples are:

The recognition of foreign proteins or foreign biopolymers by soluble antibodies or T-cell receptors.

Pathogen surface molecules binding to cellular receptors (e.g. adhesion molecules) to gain access to the cytoplasm.

The binding of cytokines to cellular receptors. Cytokine binding triggers a wide range of signal transduction events in a cell. For instance in tumor metastasis, angiogenesis and tumor cell proliferation is promoted.

The binding of fibronectin by integrins to affect blood clotting.

The binding of biological targets by small organic pharmaceutical molecules.

The manufacture of diagnostic and therapeutic agents, including small molecules, peptides, and monoclonal antibodies that likewise recognize and bind to specific bio-molecular targets, is the basis of the pharmaceutical industry. A new class of materials capable of high affinity binding to a broad range of therapeutically and diagnostically important targets, and that display better selectivity, lower side effects, and better stability could find widespread use.

Aqueous reverse microemulsions, which are surfactant aggregates in nonpolar solvents that enclose packets of aqueous solution, have been widely studied experimentally. They are discussed, for example, in Microemulsion Systems, edited by H. L. Rosano and M. Clausse, New York, N.Y.: M. Dekker, 1987, and in Handbook of Microemulsion Science and Technology, edited by P. Kumar and K. L. Mittel, New York, N.Y.: M. Dekker, 1999. As outlined in a series of papers by Candau and co-workers (Colloid & Polymer Science, 271, 1993, 1055), blends of non-ionic surfactants can be very effective at stabilizing the polymerization of water-soluble monomers in the core of reverse microemulsions. Candau was able to stabilize mixtures of monomers and water that were 50 wt. % monomers. Very few other reported polymerizable microemulsions were able to support these high weight percentages of hydrophilic monomers and polymers.

Unilamellar liposomes are stable microscopic spherical structures consisting of a lipid bilayer surrounding an aqueous core. The lipid bilayer acts as a permeability barrier, effectively separating aqueous solutes inside and outside the liposome. The stability of liposomes has made their use attractive as drug delivery vehicles. Liposomes are capable of incorporating biomolecules into their lipid bilayer. These biomolecules include membrane-bound proteins (membrane-bound proteins are particularly important in molecular recognition). Such biomolecules are involved in molecular recognition processes and are thus potential targets of therapeutic and diagnostic materials. Virus surface molecules have also been incorporated into liposomes. Such liposomes have been referred to as “virosomes”. Polymerization of water-soluble monomers in the interior of a liposome has been sparsely reported on in the literature. One polymerization method was reported by Torchilin, et. al., Makromol. Chem., Rapid Communication, 8:457-460 (1987). Polymerization to form a crosslinked particle in the interior of a liposome may be desirable because the liposome can act as a vessel that limits the size of polymer particle. Thus, the hollow cores of liposomes have been advantageously employed to encapsulate material for a wide variety of purposes, including cosmetic and drug delivery applications. Liposomes have also been used to present membrane-bound macromolecules for diagnostic, drug delivery and drug development applications. Another use of liposomes is to form polymer spheres of nanometer dimensions in the liposome interior.

### SUMMARY OF THE INVENTION

The invention is directed to nanoparticles useful for specific non-covalent binding to biological molecular targets. More particularly, the nanoparticles of the invention are comprised of high affinity molecules incorporated into polymeric matrices. As used herein, “high affinity molecules” denotes molecules that are capable of forming strong physical (non-covalent) bonds to target molecules with association constants in the range of 10⁴ to 10¹² M⁻¹ or higher. Target templates are used to define the shape of one or more binding sites on the surface of the nanoparticle and to secure the high affinity molecule at the surface of the interface. The binding site is a three-dimensional region having a shape complementary to at least a portion of the surface of the target (also referred to herein and in the appended claims as “three-dimensional target-complementary binding sites”). The positions of the high affinity molecules in the binding sites are stabilized by a polymer network. Because the high affinity molecules of the nanoparticle have moieties that have high affinity for targets, the resulting nanoparticle will selectively bind non-covalently to the molecular targets. This selective binding is further heightened by the target-complementary shape of the binding sites. The resulting high affinity nanoparticles are hydrophilic and provide desirable attributes for successful recognition and binding of target molecules. The molecular recognition capability of these particles enables their use in research, diagnostic, therapeutic, high affinity, and separation applications. They take the form of “nano-scale articles” or “nano-articles”; that is, structures that are from about 1 to about 1000 nanometers (10⁻⁹ meters) in size.

Methods are provided for synthesizing a hydrophilic nanoparticle comprised of high affinity molecules within three-dimensional target-complementary binding sites.

The nanoparticle of the invention may be formed by contacting a “template” molecule with a set of building blocks (also referred to herein as a “building block set”) that is then polymerized and crosslinked into a “polymeric matrix” or “polymeric network” (which two terms refer to the same polymeric structure and are used interchangeably herein and in the appended claims) that contacts the template molecule. The high affinity molecule is included as one subset of the building block set. Polymerization followed by template removal yields the nanoparticle comprising a polymeric matrix having three-dimensional binding sites on the surfaces of the particle. The binding sites are complementary in shape to at least a portion of the target, and high affinity molecules are chemically anchored within the binding sites. The high affinity nanoparticle is then capable of selectively binding non-covalently to “target” molecules when presented with the target molecule in a mixture of molecules. As used herein, “templates”, “target templates”, and “targets” refer to the same molecular structure. The terms “template” and “target template” are used when discussing the nanoparticle fabrication process; whereas, the term “target” is used for example when discussing the nanoparticle in its end-use application.

The high affinity molecules in the building block set are capable of undergoing a non-covalent binding interaction with the target template and the target. The high affinity molecules also possess at least one crosslinkable group capable of covalently reacting to crosslink to other building blocks of the building block set, which other building blocks also include at least one crosslinkable group, thereby to form the polymeric network of the high affinity nanoparticle.

The high affinity nanoparticle may be formulated into appropriate forms for different routes of administration as described in the art, for example, in “Remington: The Science and Practice of Pharmacy”, Mack Publishing Company, Pennsylvania, 1995, the disclosure of which is incorporated herein by reference.

### BRIEF DESCRIPTION OF THE DRAWING

FIG. 1 is a cross-sectional view of a representative nanoparticle of the invention.

### DETAILED DESCRIPTION OF THE INVENTION

The terms “a” and “an” mean “one or more” when used herein and in the appended claims.

By “water-soluble” is meant, herein and in the appended claims, having a solubility in water of greater that 10 mg/mL, and preferably greater than 50 mg/mL.

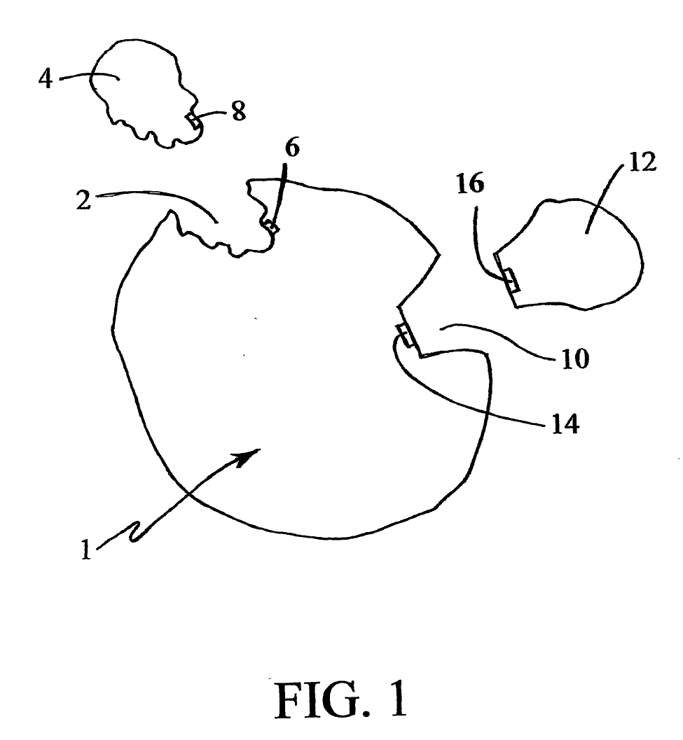

Nanoparticles comprised of high affinity molecules are provided; the nanoparticles are hydrophilic and capable of in vivo delivery. More particularly, the nanoparticles of the invention are comprised of high affinity molecules incorporated into polymeric matrices. Target templates are used to define the shape of one or more binding sites of the nanoparticle and to secure the high affinity molecule into position at the surface of the interface within the binding site during synthesis. The binding site is a three-dimensional region complementary to at least a portion of the surface of the target. The positions of the high affinity molecules in the binding sites are stabilized by a crosslinked polymer network. FIG. 1 illustrates a representative nanoparticle 1 of the invention, in cross-section. Nanoparticle 1 has, in this embodiment, two three-dimensional binding sites 2 and 10 on the surface of the particle. Binding site 2 has a shape complementary to a portion of the surface of target 4. Binding site 2 includes a high affinity molecule 6 chemically anchored on the surface therein, which high affinity molecule 6 is capable of a non-covalent binding interaction with site 8 on target 4. In like manner, binding site 10 has a shape complementary to a portion of the surface of target 12. Binding site 10 includes a high affinity molecule 14 chemically anchored therein, which high affinity molecule 14 is capable of a non-covalent binding interaction with site 16 on target 12.

By “capable of a non-covalent binding interaction” is meant that the high affinity molecule can bind non-covalently with a site on the target; this includes any high affinity molecule, either naturally-occurring or synthetic and either modified or unmodified, that has the ability or qualities necessary for binding non-covalently to a site on the target molecule. High affinity molecules which exhibit the ability to bind non-covalently to a particular site on a particular target would be known to one of skill in the art or are determinable without undue experimentation. For example, detailed parameters and discussions of therapeutic molecules with high affinity to therapeutic targets can be found, for example, in the Physician's Desk Reference, (49th Ed.), Medical Economics Data Production Co., New Jersey, 1995; the disclosure of which is incorporated by reference herein.

The high affinity molecule that forms a part of the nanoparticle of the invention can be any substance that has high affinity for a target of choice. For instance, the high affinity molecules useful in the invention may be chemical derivatives of molecules that are used to diagnose or treat disease through binding to disease-associated entities. A high affinity molecule may range in size from a small molecule, e.g., with a size less than 1 nm, to a biological macromolecule such as a protein with a size of up to 10 nm. The high affinity molecule may be, for example, a small molecule such as a drug (e.g., doxorubicin, serotonin, and the like), a peptide (such as an RGD peptide or an epitope of a receptor-binding protein, e.g.), or a protein (IL-1, IL-2, and IFNα being just a few examples).

The high affinity molecule is functionalized with a crosslinkable group to allow chemical anchoring to the polymeric network of the nanoparticle. By chemically anchoring a high affinity molecule on the surface or the interface within the well of the binding site of a nanoparticle, the resulting high affinity nanoparticle will exhibit improved or otherwise desirable properties such as increased target selectivity, higher target binding strength, and decreased toxicity.

The nanoparticles of the present invention may comprise one high affinity molecule or a mixture of high affinity molecules. If a mixture, the mixture may be present as a physical mixture of discrete separate nanoparticles. A single nanoparticle may include two or more high affinity molecules anchored on the surface within the binding sites of a nanoparticle. The nanoparticle may have from one to many binding sites, each having the same or different high affinity molecules therein.

The nanoparticles generally have a diameter of about 1000 nm or less, preferably from about 5 nm to about 400 nm, and more preferably from about 10 nm to about 200 nm. When used as therapeutics, the size of the nanoparticles will be chosen in part based upon the mode of in vivo delivery contemplated and the location of the intended target. For instance, the leaky vasculature found in tumors allows nanoparticles to leave the blood stream and concentrate in tumors. This effect, described as enhanced permeability and retention (EPR) for macromolecular agents, has been observed to be universal in solid tumors (H. Maeda, et. al., J. Controlled Release, 2000, V65, p.271-284). The key mechanism for the EPR effect for macromolecules is retention, whereas low-molecular-weight substances are not retained but are returned to circulating blood by diffusion. The size of nanoparticles may be optimized from 5 to 100 nm for accumulation in solid tumors (large enough to take advantage of the EPR effect, while not being so large that passage out of the blood vessels is overly impeded.

The nanoparticles are constructed from building blocks. Building blocks are molecular species that possess crosslinkable moieties. The high affinity molecule is one building block in a set of building blocks. The majority of the building blocks do not possess high affinity to an intended target, or are not used for their bioactive properties. Rather, they are used to make the scaffold of the nanoparticle. They are referred to herein as “scaffolding building blocks”. In one embodiment of the invention, the building block scaffold that contacts the target provides a substantial increase in binding selectivity to the target.

In one method, the nanoparticles of the present invention are formed in the interior of reverse microemulsions. Essentially, the nanoparticle is formed by contacting a target template molecule with building blocks (some of which are high affinity molecules) solubilized in the aqueous core of a reverse microemulsion. The reverse microemulsion structure is supported through the use of certain surfactants. Surfactants that may be used include commercially available nonionic surfactants, such as polyoxyethylene-sorbitan (e.g. Tween®) compounds, and sorbitan alkyl ester compounds (e.g. Span®), which are available, for example, from Sigma (St Louis, Mo.). The target templates are surface-active, so that at least a portion of the template molecules locate at the oil/water interface of the reverse microemulsion. The building block solution is the dispersed phase of the reverse microemulsion and is in contact with the target template molecules at the oil/water interface, with the high affinity molecules preferably physically bound to the target. The building blocks are then polymerized to form a network that includes one or more binding sites having a complementary surface to at least a portion of the surface of the target templates. The target template is removed to produce a binding site on a nanoparticle that maps the surface of the target and that includes at least one high affinity molecule that has high affinity for the target molecule. A hydrophilic high affinity nanoparticle of similar dimensions as the reverse microemulsion that originally supported it is produced. In subsequent solutions in which the target is present, the nanoparticle is capable of molecular recognition of the target as a result of both the complementary shape of the binding site at the surface of the nanoparticle and the high affinity molecules anchored at the surface within the binding site.

In one embodiment of the reverse microemulsion process, an aqueous solution of the building blocks (some of which are high affinity molecules), surface-active target template molecules, and, in one embodiment, surfactants is added to an organic solution consisting of a hydrophobic solvent and, in one embodiment, surfactants. In this manner, a reverse microemulsion having the building blocks in the aqueous core and the target templates at the oil/water interface of the reverse microemulsion is formed. In another embodiment of the process, reverse microemulsions are formed in the organic solution from the aqueous droplets containing the building block set, and the template molecules are added thereafter. The high affinity nanoparticle prepared utilizing reverse microemulsions will be a spherical particle generally ranging in diameter from about 5 to about 500 nm, more preferably from about 10 to about 50 nm.

In another synthetic method, the high affinity nanoparticles of the present invention are formed in the interior of liposomes. The nanoparticle is formed by bringing an aqueous solution of building blocks (some of which are high affinity molecules) into contact with lipid constituents such as phosphatidyl choline and cholesterol and with target template molecules to form liposomes. The high affinity molecules bind non-covalently to moieties on the target templates, and the building blocks assemble around the target template molecule. The building blocks within the liposomes are then polymerized to form a network that at least partially surrounds the target templates. Removal of the lipid bilayer and the target template produces a cavity or well (binding site) in a nanoparticle that maps the surface of the target and includes a high affinity molecule that has high affinity for the target molecule, resulting in a hydrophilic high affinity nanoparticle sphere of the same dimensions as the liposome that contained it and being capable of molecular recognition of the target.

In one embodiment of the liposome synthesis process, liposomes are formed with the template molecules attached to the bilayer via linkage to a hydrophobic moiety prior to the addition of the building block set. In another embodiment of the process, liposomes are formed from the building blocks and the lipids, and the template molecules are added thereafter. In a third embodiment, the lipids and the target template molecules are all added together and liposomes are then formed. The high affinity nanoparticles prepared utilizing liposomes will generally range in diameter from about 20 to about 1000 nm, more preferably from about 25 to about 250 nm. The size can be controlled through well-known liposome processing techniques such as extrusion prior to polymerization.

Provided in one embodiment are compositions comprising the high affinity nanoparticle in a pharmaceutically acceptable carrier. Pharmaceutical carriers suitable for a particular route of administration may be used. Exemplary routes of administration include orally, parenterally, topically, intravenously, and by inhalation, implantation, mucosal delivery, dermal delivery, and ocular delivery. The nanoparticle may be formulated into appropriate forms for different routes of administration as described in the art, for example, in Remington: The Science and Practice of Pharmacy, Mack Publishing Company, Pennsylvania, 1995; the disclosure of which is incorporated herein by reference.

Building Block Sets

The building block set comprises high affinity molecules together with other building blocks. The building block set may include, for example, at least about 1 to about 50, e.g. about 2 to 10, different types of building blocks in addition to the high affinity molecules. The building block set must be water-soluble for incorporation into the aqueous core of the reverse microemulsion or the liposome. Where synthesis of the nanoparticle is by reverse emulsification, the building block set must not disrupt the structure of the reverse microemulsion to the extent that the production of nanoparticles is substantially hindered. When synthesis of the nanoparticle is by use of liposomes, the building block set must not disrupt the structure of the liposome. Disruption of the liposome bilayer may occur if the building block set is either too “organic”, i.e. has a hydrophobic character, or too surface-active in nature. In this case, the lipids will not self-assemble in a bilayer, but will instead be somewhat soluble in the aqueous building block solution. Also, to prevent building blocks from leaking out of the liposome core prior to crosslinking, the time scale for building blocks to diffuse through the lipid bilayer should be slow compared to the time scale of nanoparticle fabrication.

In one preferred embodiment, the building block set includes a plurality of different building blocks, with the same crosslinkable group. The set may include two, or optionally three or more different types of building blocks, in addition to the high affinity molecules. More complex sets may be designed which have about 4-6, or about 7-10 different building blocks, or optionally about 10-20, or 20 or more different building blocks. The selection and ratio of building blocks in a set may be designed selectively for a particular target.

The types, number and relative amounts of the building blocks in a set thus can depend on the nature of the target. For example, if the target has a high density of negative charges, the building block set may include a large number of positively charged building blocks, and vice versa. Additionally, hydrogen bond donors on the target would suggest complementary hydrogen bond acceptors. An important consideration in the selection of the building blocks is the diversity needed to effectively map the regions of interest on the target surface. As an example, most protein surfaces are populated with charged residues such as lysine (R—NH₃⁺) or arginine (R—C(NH₂)₂⁺). Thus, the building blocks are in one embodiment provided with anionic counterparts such as carboxylates (R—CO₂⁻) and sulfates (R—SO₃⁻). Anionically charged protein surface residues such as glutamate (R—CH₂—CH₂—CO₂⁻) or aspartate (R—CH₂—CO₂⁻) are complexed by complementary cationic moieties such as ammonium (R—NR₃⁺) or amidines (R—C(NR₂)₂⁺).

Hydrophobic dehydration of the protein surface can result in large binding energetics. Thus, building blocks that produce a nanoparticle structure that results in the dehydration of the protein surface can be used. Such building blocks should be of intermediate polarity. They should be hydrophilic enough to be soluble in the water core of a reverse microemulsion, for example, but hydrophobic enough to promote hydrophobic dehydration of the protein. Sugar moieties may be particularly attractive for this purpose.

Building Blocks

Building blocks can have a monomeric, oligomeric, or polymeric structure. In one embodiment, building blocks are generally comprised of i) moieties that are complementary to a template surface, and ii) functional (crosslinkable) moieties that allow the building blocks to be covalently crosslinked to one another. Building blocks are generally hydrophilic water-soluble compounds. In the practice of the present invention, building blocks are usually utilized as building block sets.

Example Building Blocks

A. High Affinity Building Blocks

The high affinity building blocks comprise a high affinity molecule having i) at least one high affinity group, which is a functional group capable of a non-covalent, and preferably specific, binding interaction with a site on the target template, and ii) at least one crosslinkable group, which is a functional group capable of undergoing a covalent reaction with another of the building blocks. The crosslinkable group preferably permits crosslinking and/or polymerization of the high affinity molecules with other building blocks under certain conditions. If the high affinity molecule does not include crosslinkable groups, the molecule may be chemically modified, by methods known in the art, to add such crosslinkable groups.

The high affinity building block may be comprised of a natural or synthetic molecule or structure. Examples include amino acids, peptides, proteins, glycoproteins, organic compounds, active agents, natural or synthetic drugs, steroids and steroid derivatives, saccharides, polysaccharides, lipopolysaccharides, carbohydrates, polycations, polyanions including nucleic acids and oligonucleic acids, porphyrins and substituted porphyrins. Biological molecules or fragments thereof that function as cellular receptors, antibodies, antigens, cytokines, and enzymes may be used as high affinity building blocks.

The high affinity molecule may be comprised of any of a range of different synthetic or naturally occurring polymers, including proteins such as enzymes and antibodies and glycoproteins. The terms “protein”, “polypeptide”, and “peptide” are used interchangeably herein to refer to polymers of amino acids of any length. The polymer high affinity molecule may be polar or nonpolar, charged or uncharged. It also may be modified naturally or by intervention; for example, disulfide bond formation, glycosylation, myristylation, acetylation, alkylation, phosphorylation or dephosphorylation. Also included within the definition are polypeptides containing one or more analogs of an amino acid (including, for example, unnatural amino acids) as well as other modifications known in the art. Proteins may be obtained, for example, by isolation from natural sources or recombinantly. Exemplary proteins include, but are not limited to ceredase, calcitonin, erythropoietin, enzymes, biopharmaceuticals, growth hormones, growth factors, insulin, monoclonal antibodies, interferons, interleukins, and cytokines. Enzymes include proteases, DNAses and RNAses.

The high affinity molecule may be comprised of any of a variety of active agents, including pharmaceutical agents, biological modifiers, or diagnostic agents. Detailed parameters and discussions of active agents can be found, for instance, in the Physician's Desk Reference (1995) 49th Ed., Medical Economics Data Production Co., New Jersey.

Chemical structures of active agents may be comprised of, but are not limited to, lipids, organics, proteins, synthetic peptides, natural peptides, peptide mimetics, peptide hormones, steroid hormones, D amino acid polymers, L amino acid polymers, oligosaccharides, polysaccharides, nucleotides, oligonucleotides, nucleic acids, protein-nucleic acid hybrids, antigens and small molecules, as well as cells, tissues, cell aggregates, cell fragments. Combinations of active agents may be used. Saccharides, including polysaccharides, such as heparin, can also be included.

High affinity elements may be comprised of known peptide ligands to receptors of interest. For instance, Phoenix Peptides' peptide ligand-receptor library (http://www.phoenixpeptide.com/Peptidelibrarylist.htm) contains thousands of known peptide ligands to receptors of potential therapeutic value. Alternatively, epitopes of a protein known to have affinity to a targeted receptor may be employed. Alternatively, peptides can be found through high throughput screening of naive peptide libraries, e.g., phage display libraries or libraries of linear sequences displayed on beads, to a given protein target.

High affinity elements may be comprised of suitable steroid hormones including, but are not limited to, corticosteroids, estrogen, progesterone, testosterone and physiologically active analogs thereof. Suitable nucleic acids include, but are not limited to, DNA, RNA, and physiologically active analogs thereof.

B. Scaffold Building Blocks

Building blocks of the building block set other than the high affinity molecule building blocks are intended to provide the scaffolding of the nanoparticle upon polymerization. In the area of contact with the template, the polymeric nanoparticle will generally have a shape complementary to the template. These building blocks possess at least one crosslinkable group, which is a functional group capable of undergoing a covalent reaction with another of the building blocks. Additionally, the building blocks may include at least one functional group capable of a preferably non-covalent binding interaction with a site on the target template. Optionally, each building block may include more than one such functional group or crosslinkable group.

Exemplary monomeric building blocks include acrylamide, sodium acrylate, methylene bisacrylamide, ammonium 2,2-bisacrylamidoacetate, N-ornithine acrylamide sodium salt, N-ornithine diacrylamide, N-acryloyltris-(hydroxymethyl)methylamine, hydroxyethylacrylate, N-(2-hydroxypropyl)acrylamide, 2-sulfoethylmethacrylate, 2-methacryloylethyl glucoside, glucose monoacrylate, glucose-1-(N-methyl)acrylamide, glucose-2-acrylamide, glucose-1,2-diacrylamide, maltose-1-acrylamide, sorbitol monoacrylate, sorbitol diacrylate, sucrose diacrylate, sucrose mono(ethylenediamine acrylamide), sucrose di(ethylenediamine acrylamide), sucrose di(diethylenetriamine acrylamide), kanamycin tetraacrylamide, kanamycin diacrylamide, dextran multiacrylamide, inulin multimethacrylate, sucrose mono(ethylenediamine acrylamide) mono(diethylenetriamine acrylamide) mono(phenyl alanine) sodium salt, as well as other acrylate- or acrylamide-derivatized sugars.

Oligomeric and polymeric building blocks may be advantageously employed to produce a more stable pre-polymerized reverse microemulsion complex. By employing polymeric monomers with many copies of a given moiety, the entropy loss of assembling many monomers around a target is avoided. More favorably, one or a few multifunctional polymeric monomers are assembled around the target. Also, the strength of the interaction of the multi-functional polymer by binding multiple sites on a target can be much more stable than monomeric interactions. Another advantage of polymeric building blocks is that, compared to lower molecular weight building blocks, there may be reduced solubilization of the building blocks out of the reverse microemulsion interior. Example acrylate-functionalized polymeric building blocks include polyethyleneglycol diacrylate, chitosan with a range of acrylamide moieties, and dextran ranging in size from approximately 500 to 40,000 daltons and functionalized with a range of acrylate or acrylamide moieties and molecular weights.

C. Building Block Substituents

The building blocks can include polar or nonpolar moieties. Polar moieties for interaction with a particular target may be negatively charged, positively charged, or uncharged. Nonpolar moieties include bulky, sterically small, rigid, flexible, aliphatic and/or aromatic moieties. Uncharged polar moieties include hydrogen bond forming or non-hydrogen bond forming functional groups. Hydrogen bond-forming moieties include hydrogen bond donors or acceptors.

Exemplary moieties include alcohols, phenols, carboxylic acids, carboxylates, amides, amines, phosphates, phosphonates, sulfonates, succinates, aromatic groups including aromatic amines, ammonium salts, amidine salts, aliphatic groups, sugars, disaccharides and polysaccharides. Additional useful moieties include naturally-occurring systems and synthetic systems. For example, a naturally-occurring amino acid, an amino acid side chain, or a synthetic amino acid derivative may be included. A dimer, trimer, or oligomer of the same or different amino acid or derivative thereof may be used. Other exemplary moieties include sugars, carbohydrates, and small or large glycoproteins. Still other exemplary moieties include purines or pyrimidines, such as adenine, cytosine, guanine, and thymine.

A consideration in the selection of a building block set is the diversity necessary to complement the regions of interest on the target surface. For example, moieties of varying size, electronegativity, hydrogen-bonding tendency, hydrophobicity, etc., can be chosen. The quantitative representation of components in a building block set can be optimized for a particular complementary interaction.

Exemplary crosslinkable groups include, but are not limited to, acrylate, acrylamide, vinyl ether, epoxide, maleic acid derivative, diene, substituted diene, thiol, alcohol, amine, hydroxyamine, carboxylic acid, carboxylic anhydride, carboxylic acid halide, aldehyde, ketone, isocyanate, succinimide, carboxylic acid hydrazide, glycidyl ether, siloxane, alkoxysilane, alkyne, azide, 2′-pyridyldithiol, phenylglyoxal, iodo, maleimide, imidoester, dibromopropionate, and iodacetyl.

Preferred crosslinkable functionalities are acrylate and acrylamide moieties. Such moieties are amenable to free-radical polymerization. Free-radical polymerization can be readily achieved through the combination of U.V. light and photoinitiators, redox-coupled free radical initiators, or heat and heat-activated initiators.

The number of crosslinkable groups attached to one single building block can range, for example, from about one to ten for low molecular weight building blocks, to several hundred for polymeric building blocks. Using different amounts of building blocks from a set of building blocks with one, two, or more crosslinkable groups allows formation of networks of different tightness and topology upon crosslinking of the building blocks.

In one embodiment, there is provided a carbohydrate building block that comprises a carbohydrate region, comprising plural hydroxyl groups, wherein at least one hydroxyl group is modified to include at least one crosslinkable group. In another embodiment, at least one of the hydroxyl groups is modified to include at least one other functionality. In a further embodiment, at least one of the hydroxyl groups is modified to include at least one crosslinkable group and at least one other functionality.

The carbohydrate region of the carbohydrate building block may include a carbohydrate or carbohydrate derivative. For example, the carbohydrate region may be derived from a simple sugar, such as N-acetylglucosamine, N-acetylgalctosamine, N-acetylneuraminic acid, neuraminic acid, galacturonic acid, glucuronic acid, ioduronic acid, glucose, ribose, arabinose, xylose, lyxose, allose, altrose, apiose, mannose, gulose, idose, galactose, fucose, fructose, fructofuranose, rhamnose, arabinofuranose, and talose; a disaccharide, such as maltose, sucrose, lactose, or trehalose; a trisaccharide; a polysaccharide, such as cellulose, starch, glycogen, alginates, inulin, pullulan, dextran, dextran sulfate, chitosan, glycosaminoglycans, heparin, heparin sulfate, hyaluronates, tragacanth gums, xanthan, other carboxylic acid-containing carbohydrates, uronic acid-containing carbohydrates, lactulose, arabinogalactan, and their derivatives, and mixtures of any of these; or modified polysaccharides. Other representative carbohydrates include sorbitan, sorbitol, chitosan and glucosamine. The carbohydrate may include amine groups in addition to hydroxyl groups, and the amine or hydroxyl groups can be modified to include a crosslinking group, other functionalities, or combinations thereof.

Besides carbohydrate-based building blocks, other examples of acrylate- or acrylamide-derivatized polymeric building blocks include polyethylene glycol-based molecules, such as polyethyleneglycol diacrylate, with molecular weights ranging from 200 to 40,000 daltons.

In a preferred embodiment, to facilitate metabolism of the hydrophilic polymeric network of the nanoparticle, degradable linkages are included within the crosslinked scaffold. Degradable linkages can be included through the use of polylactide, polyglycolide, poly(lactide-co-glycolide), polyphosphazine, polyposphate, polycarbonate, polyamino acid, polyanhydride, and polyorthoester-based building blocks, among others. Additionally, degradable linkages may be used to attach polymerizable moieties to carbohydrates. Additionally, small molecule crosslinking agents containing similar hydrolyzable moieties as the polymers such as carbonates, esters, urethanes, orthoesters, amides, and phosphates may be used as building blocks. To function as degradable components in the hydrophilic scaffold, these building blocks must be functionalized with two or more polymerizable moieties. For example, polyglycolide diacrylate, polyorthoester diacrylate and acrylate-substituted polyphosphazine, acrylate-substituted polyamino acid, or acrylate-substituted polyphosphate polymers can be used as degradable building blocks. Methacrylate or acrylamide moieties can be employed instead of acrylate moieties in the above examples. Similarly, small molecules containing a hydrolyzable segment and two or more acrylates, methacrylates, or acrylamides may be used. Such degradable polymers and small molecule building blocks may be functionalized with acrylate, methacrylate, acrylamide or similar moieties by methods known in the art.

The nanoparticle polymeric network and the scaffold breakdown products of this invention are designed to be non-toxic and eliminated from the body. They may have degradable, preferably carbohydrate-based, polyamino acid-based, polyester-based, or PEG-based cores, with the rate of degradation controlled by the identity of the sugar, crosslink density, and other features. Thus, the articles can be metabolized in the body, preventing undesirable accumulation in the body.

Synthesis and Procurement of Building Blocks

The building blocks may be synthesized using methods available in the art of organic chemistry (see for example, J. March, Advanced Organic Chemistry, Fourth Ed., John Wiley and Sons, New York, Part 2, pp. 255-1120, 1992). For example, the desired functionalities and the crosslinkable groups can be coupled to a starting organic compound such as a carbohydrate using organic reactions, such as ester, amide, or ether linkage formation.

Carbohydrate-based building blocks may be prepared from the carbohydrate precursor (e.g. sucrose, sorbital, dextran, inulin, pullulan, etc.) by standard coupling technologies known in the art of bioorganic chemistry (see, for example, G Hermanson, Bioconjugation Techniques, Academic Press, San Diego, pp 27-40, 155, 183-185, 615-617, 1996; and S. Hanesian, Preparative Carbohydrate Chemistry, Marcel Dekker, New York, 1997.) For example, a crosslinkable group can be attached to a carbohydrate via the dropwise addition of acryloyl chloride to an amine-functionalized sugar. Amine-functionalized sugars can be prepared by the action of ethylene diamine (or other amines) on 1,1′-carbonyldiimidazole activated sugars. Ester-linked reactive groups can be synthesized through the reaction of acrylic or methacrylic anhydrides with the hydroxyl group of a carbohydrate such as inulin in pyridine.

Carbohydrate-based building blocks may also be prepared by the partial (or complete) functionalization of the carbohydrate with moieties that are known to polymerize under free radical conditions. For example, methacrylic esters may be placed on a carbohydrate at varying substitution levels by the reaction of the carbohydrate with methacrylic anhydride or glycidyl methacrylate (Vervoort L., et al., International Journal of Pharmaceutics, 1998, 172, 127-135).

Carbohydrate-based building blocks may also be prepared by chemoenzymatic methods (Martin B. D., et al., Macromolecules, 1992, 25, 7081), for example in which Pseudomonas cepacia catalyzes the transesterification of monosaccharides with vinyl acrylate in pyridine or by the direct addition of an acrylate (Piletsky S., et al., Macromolecules, 1999, 32, 633-636). Other functional groups may be present, as numerous derivatized carbohydrates are known to those familiar with the art of carbohydrate chemistry.

High affinity molecules will in most cases have to be functionalized with crosslinking moieties to be used as building blocks. Moieties which will act as a crosslinking site under free-radical conditions can be attached to proteins using standard coupling techniques (J. Mol. Catal. 1979, 6, 199. J. Controlled Release 1986, 4, 223.) For example, N-acryloxysuccinimide, or other derivatives which may have spacers of varying length and composition between the radical reactive moiety and the point of attachment to the protein, will react readily with amines located on the substrate protein, yielding a covalently attached, radical-reactive, crosslinking point.

Reagents and starting materials in some embodiments can be obtained commercially. For example, amino acids and purines and pyrimidines can be purchased from chemical distributors such as Aldrich (Milwaukee, Wis.), Kodak (Rochester, N.Y.), Fisher (Pittsburgh, Pa.), Shearwater Polymers (Huntsville, Ala.), Pierce Chemical Company (Rockford, Ill.) and Carbomer Inc. (Westborough, Mass.). Monomers and monomer precursors are also available commercially from Sigma Chemical Company (St. Louis, Mo.), Radcure (Smyrna, Ga.), and Polysciences (Niles, Ill.). Additionally chemical product directories and resources such as<http://www.chemdex.com> and <http://pubs.acs.org/chemcy/> may be used to locate starting materials.

Targets

Nanoparticles may be formed with a high and specific binding affinity for any of a variety of targets. Where the nanoparticles are synthesized via reverse microemulsion, the primary requirement for a target is that it must be amphiphilic, either in its natural state or by chemical modification, in order to keep the target, as the template, at the oil/water interface of the reverse microemulsion during nanoparticle formation.

A target may range in size from a small molecule, e.g., with a size less than 1 nm, to a biological macromolecule such as a protein with a size of up to and greater than 10 nm. The target may be a molecule, or a portion of a molecule, such as the Fc region or the epitope portion of an antibody. The target may be a complex biological structure such as a virus or a portion of a virus, a bacterium or a portion of a bacterium, a eukaryotic cell surface or a portion of a eukaryotic cell surface. The target also may be an inorganic nanostructure or a microstructure.

The target may be a natural or synthetic molecule or structure. Examples include organic compounds, toxins, natural and synthetic drugs, steroids and steroid derivatives. Biopolymers are preferred targets, and include proteins, glycoproteins, saccharides, polysaccharides, lipoproteins, lipopolysaccharides, and oligonucleotides. Preferred targets are biological molecules which function as cellular receptors, cytokines, and growth factors.

The terms “protein”, “polypeptide”, and “peptide” are used interchangeably herein to refer to polymers of amino acids of various lengths. The target may be polar or nonpolar, charged or uncharged. The target may be linear, branched, folded, or aggregated. It may comprise modified amino acids, and it may be interrupted by non-amino acids. It also may be modified naturally or by intervention; for example, disulfide bond formation, glycosylation, myristylation, acetylation, alkylation, phosphorylation or dephosphorylation. Also included within the definition are polypeptides containing one or more analogs of an amino acid (including, for example, unnatural amino acids) as well as other modifications known in the art.

The target may be any of a variety of active agents, including pharmaceutical agents, biological modifiers, or diagnostic agents. Detailed parameters and discussions of active agents can be found, for instance, in the Physician's Desk Reference (1995) 49th Ed., Medical Economics Data Production Co., New Jersey.

The chemical structures of active agents include, but are not limited to, lipids, organics, proteins, synthetic peptides, natural peptides, peptide mimetics, peptide hormones, steroid hormones, D amino acid polymers, L amino acid polymers, oligosaccharides, polysaccharides, nucleotides, oligonucleotides, nucleic acids, protein-nucleic acid hybrids, antigens and small molecules, as well as cells, tissues, cell aggregates, cell fragments. Combinations of active agents may be used. Saccharides, including polysaccharides, such as heparin, can also be included.

Proteins, protein fragments or peptides may be obtained, for example, by isolation from natural sources, recombinantly, or through solid state synthesis. Examples include, but are not limited to, cytokines such as interferons, interleukins and TNF-a; growth hormones; growth factors such as EGF and VEGF; growth factor receptors such as VEGFR-2; erbB-1, erbB-2; insulin; erythropoietin; monoclonal antibodies; fibrin, collagen and other extracellular matrix molecules; and enzymes such as proteases, DNAses and RNAses.

Suitable steroid hormones include, but are not limited to, corticosteroids, estrogen, progesterone, testosterone and physiologically active analogs thereof. Suitable nucleic acids include, but are not limited to, DNA, RNA and physiologically active analogs thereof.

Specific examples of active agents are listed in U.S. patent application Ser. No. 09/172,921, the entire disclosure of which is incorporated by reference herein.

Synthesis of Hydrophilic High Affinity Nanoparticles

The nanoparticles of the present invention can be formed in one of several ways, with the exact procedure for nanoparticle formation being determined by features such as building block solubility, chemical composition of the high affinity molecule(s) and/or the target template molecule(s), the desired size of the resulting nanoparticle, and the intended use of the nanoparticle.

Overview of Nanoparticle Formation Using Reverse Microemulsions

Water, building blocks (including high affinity molecules), target templates, and surfactants are added to an organic solvent to form a reverse microemulsion having target templates incorporated into the interface between the reverse microemulsion and the solvent, the interface surrounding and enclosing an aqueous solution containing nanoparticle building blocks solubilized at about 5 wt % to about 75 wt %. The building blocks are then polymerized, following standard polymerization procedures. The organic solvent, non-reactive surfactants, and the target templates are thereafter removed to give a hydrophilic nanoparticle of the same dimensions as the reverse microemulsion that contained it and having three-dimensional surface sites complementary to targets. Surfaces of the high affinity molecules are located in the sites, which surfaces include functional groups that are complementary to surface sites of the target molecules, resulting in the ability of the nanoparticles to selectively bind to the targets. It is contemplated that from 1 to about 100 recognition sites may be present on the nanoparticle, to selectively bind to from 1 to about 100 target molecules, which targets may be the same or different.

Detailed Description of Nanoparticle Formation Using Reverse Microemulsions

Reverse microemulsions are formed in an organic solvent as small droplets of aqueous solution containing water-soluble nanoparticle building blocks by methods well known to those practiced in the art. They are discussed, for example, in Microemulsion Systems, edited by H. L. Rosano and M. Clausse, New York, N.Y.: M. Dekker, 1987, and in Handbook of Microemulsion Science and Technology, edited by P. Kumar and K. L. Mittel, New York, N.Y.: M. Dekker, 1999.

In one embodiment, an aqueous solution comprising a set of solubilized building blocks, including high affinity molecules, is contacted with an organic solvent comprising one or more surfactants to form a reverse microemulsion having the building blocks concentrated within the reverse microemulsion. Target templates are added. Because these templates are amphiphilic, they will tend to locate at the interface between the organic solvent and the aqueous core. A portion of the template will be in contact with the aqueous building block solution in the core of the microemulsion nanodroplets.

Surfactants are utilized to stabilize the reverse microemulsion. These surfactants do not include crosslinkable moieties; they are not building blocks. Surfactants that may be used include commercially available surfactants such as sorbitan esters including sorbitan monooleate (Span® 80), sorbitan monolaurate (Span® 20), sorbitan monopalmitate (Span® 40), sorbitan monostearate (Span® 60), sorbitan trioleate (Span® 85), and sorbitan tristearate (Span® 65), which are available, for example, from Sigma (St Louis, Mo.). Sorbitan sesquioleate (Span® 83) is available from Aldrich Chemical Co., Inc. (Milwaukee, Wis.). Other surfactants which may be used include polyoxyethylenesorbitan (Tween®) compounds. Exemplary cosurfactants include polyoxyethylenesorbitan monolaurate (Tween® 20 and Tween® 21), polyoxyethylenesorbitan monooleate (Tween® 80 and Tween® 80R), polyoxyethylenesorbitan monopalmitate (Tween® 40), polyoxyethylenesorbitan monostearate (Tween® 60 and Tweene 61), polyoxyethylenesorbitan trioleate (Tween® 85), and polyoxyethylenesorbitan tristearate (Tween® 65), which are available, for example, from Sigma (St Louis, Mo.).

Other exemplary surfactants include fatty acid soaps, alkyl phosphates and dialkylphosphates, alkyl sulfates, alkyl sulfonates, primary amine salts, secondary amine salts, tertiary amine salts, quaternary amine salts, n-alkyl xanthates, n-alkyl ethoxylated sulfates, dialkyl sulfosuccinate salts, n-alkyl dimethyl betaines, n-alkyl phenyl polyoxyethylene ethers, n-alkyl polyoxyethylene ethers, sorbitan esters, polyethyleneoxy sorbitan esters, sorbitol esters and polyethyleneoxy sorbitol esters.

Other exemplary commercially available surfactants include polyethyleneoxy(40)-sorbitol hexaoleate ester (Atlas G-1086, ICI Specialties, Wilmington Del.), hexadecyltrimethylammonium bromide (CTAB, Aldrich), polyethyleneoxy(n)nonylphenol (Igepal™, Rhone-Poulenc Inc. Surfactants and Specialties, Cranbrook, N.J.), and linear alkylbenzene sulfonates (LAS, Ashland Chemical Co., Columbus, Ohio).

Other surfactants include lipids, such as phospholipids, glycolipids, cholesterol and cholesterol derivatives. Exemplary lipids include fatty acids, or molecules comprising fatty acids, wherein the fatty acids include, for example, palmitate, oleate, laurate, myristate, stearate, arachidate, behenate, lignocerate, palmitoleate, linoleate, linolenate, and arachidonate, and salts thereof such as sodium salts. The fatty acids may be modified, for example, by modification of the acid functionality to the sulfonate by a chain extension reactions known in the art.

Cationic lipids may be used as cosurfactants, such as cetyl trimethylammonium bromide/chloride (CTAB/CTAC), dioctadecyl dimethyl ammonium bromide/chloride (DODAB/DODAC), 1,2-diacyl-3-trimethylammonium propane (DOTAP), 1,2-diacyl-3-dimethyl ammonium propane (DODAP), [2,3-bis(oleoyl)propyl] trimethyl ammonium chloride (DOTMA), and [N-(N′,N′-dimethylaminoethane)-carbamoyl]cholesterol, dioleoyl) (DC-Chol).

Phospholipids which may be used also include phosphoglycerides, such as phosphatidyl cholines. Lipids developed in the art of gene delivery also may be used, as described, for example, in Lasic, “Liposomes in Gene Delivery”, CRC Press, New York, 1997; and U.S. Pat. No. 5,459,127, the disclosures of which are incorporated herein by reference. Examples include N-[1-(2,3-dioleoxy)propyl]-N,N,N-trimethyl ammonium chloride (DOTMA), and dimyristooxypropyl dimethyl hydroxyethyl ammonium bromide (DMRIE). Other lipids include sphingosine.

Crosslinking

After the reverse microemulsion system is formed, the crosslinkable groups of the building blocks are preferably reacted in order to crosslink the building blocks. Crosslinking of the building blocks in the reverse microemulsion interior can be initiated using standard polymerization procedures well known to those practiced in the art (see, for example, Odian G. G.; Principles of Polymerization, 3rd Ed., Wiley, New York, 1991; L. H. Sperling, Introduction to Physical Polymer Science, Chapter 1, pp.1-21, John Wiley and Sons, New York, 1986; and R. B. Seymour and C. E. Carraher, Polymer Chemistry, Chapters 7-11, pp.193-356, Dekker, New York, 1981). For instance, if the building blocks possess functional/crosslinkable groups amenable to free radical polymerization such as acrylates and acrylamides, polymerization can be induced through the combination of U.V. initiators and U.V. light or thermal initiators and heat.

Nanoparticle Isolation

After the assembled building blocks are crosslinked to form the nanoparticle, it is necessary to remove the oil phase and surfactant molecules surrounding the polymerized nanoparticle. The target template is also released in this step or subsequently released, and the nanoparticle is isolated.

The surfactants and the oil phase, residual from the synthesis of the nanoparticle core, can be removed through the use (singularly or in combination) of solvent washing, for instance using ethanol to solubilize the surfactant and oil while precipitating the polymer nanoparticles; surfactant-adsorbing beads; dialysis; or the use of aqueous systems such as 4M urea. Methods for surfactant removal are known in the art.

To release a target template, the physical interactions may be dampened or reversed, or the target's morphology may be altered. This can be implemented by, for example, altering the pH, the ionic strength, the solvent or the temperature of the template-nanoparticle complex. Electrostatic interactions can be dampened by increasing the ionic strength. Altering the pH, using urea, or raising the temperature all can be used to alter the charge and charge distribution of a target such as a biomolecule, or to denature a template protein. With these changes, the noncovalent interactions between the template and the nanoparticle are disrupted, causing the template to be released. Alternatively, proteases that will digest or break down a peptide target may be added to the solution. Conditions are selected such that the nanoparticle remains essentially undamaged. For example, the nanoparticle may have enhanced stability due to its covalently crosslinked and hydrolytically-stable chemical structure.

After the template molecules have been detached from the high affinity nanoparticles, the high affinity nanoparticles can be purified, for example by standard dialysis or chromatographic separations well-documented in the protein separation art.

Overview of Nanoparticle Formation Using Liposomes

Lipids, building blocks (including high affinity molecules), and target templates are mixed together to form a liposome having target templates incorporated into the lipid bilayer of the liposome, the lipid bilayer surrounding and enclosing an aqueous solution containing the water-soluble building blocks. The building blocks are then polymerized, following standard polymerization procedures. The lipid bilayer and the target templates are thereafter removed to give a hydrophilic high affinity nanoparticle of the same dimensions as the liposome that contained it and having three-dimensional surface sites that map at least a portion of the surface of the target templates. Surfaces of the high affinity molecules are located in the sites, which surfaces include functional groups that are complementary to surface sites of the target molecules, resulting in the ability of the high affinity nanoparticles to selectively bind to the targets. It is contemplated that from 1 to about 10,000, preferably about 10 to about 1000, recognition sites may be present on the nanoparticle that selectively bind to target molecules, which targets may be the same or different than the template molecules used to create the binding site.

Detailed Description of Nanoparticle Formation Using Liposomes

Liposomes are formed in an aqueous solution containing water-soluble nanoparticle building blocks, including high affinity molecules. The building blocks must not inhibit the formation of the lipid bilayer. This can be accomplished by using highly water-soluble building blocks, and by producing a highly hydrophilic polymer product.

Liposomes can generally be formed from an aqueous solution containing building blocks and lipids by methods well known to those practiced in the art. A detailed discussion of various liposome forming methods is given in Liposomes: a Practical Approach, R. R. New, Ed., Oxford University Press, New York, 1990. Such methods include sonication, extrusion, detergent depletion, and reverse phase evaporation.

In one embodiment, liposome formation may proceed as follows. The lipid constituents are solvated in an organic solvent such as diethyl ether or chloroform. Evaporating off the organic solvent in a round bottom flask then forms a lipid film. The aqueous building block solution can then be introduced to the flask and, with stirring, liposomes are formed. This solution can contain the target template, or the template can be added in an ensuing step. A broad size dispersion of liposomes is produced in this manner. Extrusion of this liposome solution can be used at this point to resize the liposomes to well-defined diameters.

Lipids that may be employed for liposome formation are those generally used in the art and include, but are not limited to, phosphatidylcholines, phosphatidylserines, phosphatidylglycerols, phosphatidylethanolamines, phosphatidylinositol, sphingophospholipids, sphingoglycolipids, as well as synthetic lipids. Mixtures of the lipids, as well as additional components that reside in the bilayer, such as cholesterol and single-chain detergents such as sodium dodecyl sulphate, may be employed. Single chain detergents are known to aid in the inclusion of membrane-bound proteins to the lipid bilayer (Liposomes: From Physics to Applications, D. Lasic, Elsevier, Amsterdam, 1994).

An alternative way to locate lipid bilayer-bound target templates in the liposome prior to polymerization is to form liposomes with the target template, but not in the presence of the building blocks. This method may be advantageous in some systems because the target template materials may be incorporated into liposomes in purification steps. The separation and purification of membrane-bound target templates, such as proteins, from the cellular mass, followed by reconstitution into liposomes is well described in the art. Typically, a detergent such as octylglycoside, octethyleneglycol dodecyl monoether (C₁₂E₈), tetraethyleneglycol octyl monoether (C₈E₄), or Triton X100 (chemically octylphenol poly(ethylengycoether)), is used with an amphiphilic protein to aid in aqueous dissolution and to aid in liposome incorporation (D. Lasic, Liposomes, pp. 244-247, supra). Individual liposome structures comprising both the target template and building blocks can then be obtained by extruding a mixture of a liposome-building block solution and liposomes containing target templates. Alternatively, the detergent/protein complex may be added during the liposome-building block formation.

The lipid bilayer of a liposome provides an excellent format for locating an amphiphilic target template at the surface of a building block solution. Targets amenable to incorporation into the lipid bilayer are precisely those that are important as diagnostic and high affinity targets. That is, eukaryotic cellular surface receptors, such as those important for signal transduction events in tumor cell metastasis, pathogen entry, metabolism, wound healing, immune response, neurotransmission, osteoporosis, rheumatoid arthritis, as well as many other biological and physiological events, are naturally located at lipid bilayers. Generally, eukaryotic transmembrane proteins, apolipoproteins, lipid-linked proteins, lipopolysaccarides, and gram-negative bacteria receptor proteins and endotoxin may be directly incorporated into the lipid bilayer. Other ligands that may be used as templates include antibodies, cytokines, peptides, glycoproteins, and pathogen toxins. Hydrophilic biomolecules with low surface activity can be presented at the liposome surface by attachment of a hydrophobic “anchor”. One possible chemical modification method is to functionalize the biomolecule with a lipid tail. As an example, a method well known to those skilled in the art is the reaction of a surface amine on a soluble protein (for example the amine terminus or a lysine residue) with an activated ester on a molecule that also is comprised of one or more hydrophobic tails (see for example Bionanoparticle Techniques, G. T. Hermanson, pp. 556-570).

After liposome formation, there are building blocks both inside and outside the liposomes. To prevent polymerization outside of the liposome, building blocks are removed from outside the liposome, for example by running the liposome solution through a gel permeation chromatography (GPC) column. The liposome structure is preserved by eluting the column with a nonreactive solution with an osmolality equal to or greater than the interior of the liposome. Using a solution with greater osmolality (higher solute concentration) will result in the dehydration of the liposome interior. The osmotic potential of the eluting solvent can be controlled through the concentration of solutes such as glucose or sodium chloride. After elution, the building block solutions contained in the liposomes can be polymerized.

Crosslinking

Crosslinking of the building blocks in the liposome interior can be initiated using standard polymerization procedures well known to those skilled in the art (see, for example, Odian G. G.; Principles of Polymerization, 3^{rd} Ed., Wiley, New York, 1991). For instance, if the building blocks possess functional/crosslinkable groups amenable to free radical polymerization such as acrylates and acrylamides, polymerization can be induced through the combination of UV initiators and UV light or thermal initiators and heat. Generally, solubility decreases as polymerization proceeds. Thus, precipitation and, perhaps, liposome destruction can occur should the water solubility of the forming nanoparticle decrease too much, and care should be taken in choosing building blocks. If the nanoparticles are to be used internally as therapeutics, they should be degradable in the body to benign materials. The materials should degrade on a time scale consistent with efficacious therapy. If the nanoparticles are to be used ex vivo for separation/purification applications, they should be engineered to be robust and resistant to degradation (i.e., fewer amide and ester linkages).

Nanoparticle Isolation

After the assembled building blocks are crosslinked to form the nanoparticle, it is necessary to remove the lipid molecules surrounding the polymerized nanoparticle. The target template is also released in this step or subsequently released, and the nanoparticle is isolated.

The lipid molecules can be removed through the use (singularly or in combination) of surfactant-adsorbing beads, dialysis, solvent washing, or the use of aqueous systems such as 4M urea. Methods for lipid removal are known in the art.

To release a target template, the physical interactions may be dampened or reversed, or the target's morphology may be altered. This can be implemented by, for example, altering the pH, the ionic strength, the solvent or the temperature of the template-nanoparticle complex. Electrostatic interactions can be dampened by increasing the ionic strength. Altering the pH, using urea, or raising the temperature all can be used to alter the charge and charge distribution of a target such as a biomolecule, or to denature a template protein. With these changes, the noncovalent interactions between the template and the nanoparticle are disrupted, causing the template to be released. Alternatively, proteases that will digest or break down a peptide target may be added to the solution. Conditions are selected such that the nanoparticle remains essentially undamaged. For example, the nanoparticle may have enhanced stability due to its covalently crosslinked and hydrolytically-stable chemical structure.

### EXAMPLES

### Example 1

### Example Reverse Microemulsion System

Nonionic surfactants are useful in solubilizing high aqueous concentrations of ionic monomers in reverse microemulsions. See for example, Candau et. al., Colloid & Polymer Science, 271, 1993, 1055. In general, an oil phase is prepared containing mixtures of Span-80 and Tween 80. For Isopar M (Exxon Co. USA, Houston Tex.) (a C₁₄-C₁₅ aliphatic oil) as the continuous phase, about 10-100 mM of each surfactant (approximately 2.5-25 wt. % surfactant blend in oil) is needed, for example, for miscibility. When less surfactant is used, complex phase separation can be observed. The direct injection method allows the co-solubilization of polar monomers in the aqueous phase. A reverse microemulsion is typically formed by adding an aqueous phase to a surfactant-containing oil phase. The aqueous phase is comprised of buffer and scaffolding building blocks such as acrylamide at 5-50 wt %, preferably 10-50 wt %, most preferably 20-40 wt %, glucose 2-acrylamide at 5-50 wt %, preferably 10-50 wt %, and ammonium 2,2-bisacrylamidoacetate at 1-40 wt %. The target and the high affinity building blocks are also incorporated into the aqueous building block solution prior to direct injection at concentrations of 0.5 to 5 wt % of the aqueous solution. If the target is a protein, it may be modified by attaching a hydrophobic tail to make it more surface-active. High affinity molecules will in most cases have to be functionalized with crosslinking moieties to be used as building blocks. For example, to attach free-radical moieties to proteins, standard coupling techniques reacting NHS-functionalized acryloyl molecules with lysine groups on the surface of the protein can be employed. After formation of the reverse microemulsion, oxygen is removed from the system and the building blocks are then crosslinked via a UV initiator in combination with UV-irradiation to form the high affinity nanoparticle.

### Example 2

### Example Reverse Microemulsion System

An oil phase is prepared containing mixtures of Span-80 and Tween 80 in Isopar M (Exxon Co. USA, Houston Tex.) (a C₁₄-C₁₅ aliphatic oil). about 10-100 mM of each surfactant (approximately 2.5-25 wt. % surfactant blend in oil) is needed, for example, for miscibility. A reverse microemulsion is then formed by adding an aqueous phase to the surfactant-containing oil phase. The aqueous phase is comprised of acrylamide at 10 wt %, glucose 2-acrylamide at 15 wt %, and ammonium 2,2-bisacrylamidoacetate at 5 wt %. The target, palmitoyl-sEGFR and 1 wt % of high affinity building blocks comprised of the amino acid sequence KGGGYCPIWKFPDEECY, where the N-terminal lysine has been functionalized with an acryloyl moiety through the reaction of methacrylic anhydride are also incorporated into the aqueous building block solution prior to direct injection at concentrations of 0.5 to 5 wt % of the aqueous solution. After formation of the reverse microemulsion, oxygen is removed from the system and the building blocks are then crosslinked using Eosin as an initiator (0.1 wt % of acrylamide) in combination with UV-irradiation to form the high affinity nanoparticle.

### Example 3

### Example Liposome System

A lipid dry film is formed by rotary evaporating a phosphatidylcholine/cholesterol/chloroform solution. The lipid phase is hydrated by adding filtered PBS buffer to the flask, and the flask is agitated gently until a cloudy homogeneous suspension is obtained. A solution (5-10 mL) of a protein-lipid conjugate/cholate as the target template is then added. Note, the lipid-functionalized target template is prepared using standard coupling techniques and is 4-5 mg PE-template per mL and 20 mg sodium cholate/mL. The cholate is removed via dialysis, after which the solution is lyophilized into a thin layer by gently rotating the round bottom in a liquid nitrogen bath.

An aqueous phase comprised of high affinity peptide building blocks at 1-5 wt %, scaffold building blocks such as glucose-2 acrylamide at 5-40 wt %, sodium ornithine diacrylamide at 1-40 wt %, and a UV-photoinitiator (such as 4,4′-azobis(4-cyanovaleric) acid) is added to the lyophilized lipid-functionalized target templates. Note that the selected building block monomers must be capable of being encapsulated in the interior of liposomes. Small monomers, such as acrylamide, and hydrophobic monomers, such as methylenebisacrylamide (MBA), show significant leakage from liposomes and are not suitable for this system.

The lipids are allowed to fully hydrate in the aqueous solution. A desired ratio is 5 mL aqueous phase/250 mg lipid phase. Use freeze-thaw cycles to fully hydrate the lipid bylayers. The hydrated lipid sample is hydrated through 100 or 400 nm pore diameter polycarbonate membranes to produce the desired liposome size. Extra-liposomal monomers are removed by gel permeation chromatography (GPC), and the liposomes are eluted with approximately 60 mL of an osmoregulating buffer. Oxygen can be removed from the liposome solution using a N₂/water aspirator line. Irradiation of the sample with UV light produces the high affinity polymeric nanoparticles.

### Example 4

### Example Liposome System

A lipid dry film is formed by rotary evaporating a phosphatidylcholine/cholesterol/chloroform solution. The lipid phase is hydrated by adding filtered PBS buffer to the flask, and the flask is agitated gently until a cloudy homogeneous suspension is obtained. A solution (5-10 mL) of a protein-lipid conjugate/cholate as the target template is then added. Note, the lipid-functionalized target template is prepared using standard coupling techniques and is 4-5 mg PE-template per mL and 20 mg sodium cholate/mL. The cholate is removed via dialysis, after which the solution is lyophilized into a thin layer by gently rotating the round bottom in a liquid nitrogen bath.

An aqueous phase comprised of high affinity peptide used in Example 2 at 1 wt %, scaffold building blocks glucose—2 acrylamide at 15 wt %, sodium ornithine diacrylamide at 4 wt %, and the initiator 4,4′-azobis(4-cyanovaleric) acid at 0.01 wt % of the aqueous phase is added to the lyophilized lipid-functionalized target templates. Note that the selected building block monomers must be capable of being encapsulated in the interior of liposomes. Small monomers, such as acrylamide, and hydrophobic monomers, such as methylenebisacrylamide (MBA), show significant leakage from liposomes and are not suitable for this system.

The lipids are allowed to fully hydrate in the aqueous solution. A desired ratio is 5 mL aqueous phase/250 mg lipid phase. Use freeze-thaw cycles to fully hydrate the lipid bylayers. The receptor human EGFR is added to the mixture. The hydrated lipid sample is then pressed through 100 or 400 nm pore diameter polycarbonate membranes to produce the desired liposome size. Extra-liposomal monomers are removed by gel permeation chromatography (GPC), and the liposomes are eluted with approximately 60 mL of an osmoregulating buffer. Oxygen can be removed from the liposome solution using a N₂/water aspirator line. Irradiation of the sample with UV light produces the high affinity polymeric nanoparticles. Removal of the target template is accomplished by denaturing the target with 4 M urea.

Although the foregoing invention has been described in some detail by way of illustration and example for purposes of clarity and understanding, it will be apparent to those skilled in the art that certain changes and modifications may be practiced. Therefore, the description and examples should not be construed as limiting the scope of the invention.

What is claimed is:

1. A hydrophilic polymeric nanoparticle comprising:
one or more three-dimensional binding sites, said binding sites being complementary in shape to at least a portion of the surface of a target; and
one or more high affinity molecules chemically anchored at the surface within said binding sites, said high affinity molecules being capable of a non-covalent binding interaction with a site on said target.

2. A nanoparticle according to claim 1 wherein said high affinity molecule is natural or synthetic and is selected from the group consisting of amino acids, peptides, polypeptides, proteins, glycoproteins, saccharides, polysaccharides, carbohydrates, lipopolysaccharides, nucleic acids, oligonucleic acids, porphyrins, substituted porphyrins, polyanions, polycations, organic compounds, steroids, steroid derivatives, drugs, enzymes, antibodies, antigens, cytokines, cellular receptors, cellular receptor fragments, and active agents.

3. A nanoparticle according to claim 1 having a diameter of from about 5 nm to about 400 nm.

4. A nanoparticle according to claim 1 having a diameter of from about 5 nm to about 200 nm.

5. A nanoparticle according to claim 1 having a diameter of from about 5 nm to about 100 nm.

6. A nanoparticle according to claim 1 wherein the position of said high affinity molecule in said binding site is stabilized by a polymer network.

7. A hydrophilic nanoparticle comprising a crosslinked three-dimensional polymeric network, said polymeric network being formed from i) scaffolding building blocks and ii) one or more high affinity building blocks comprising a high affinity molecule, said polymeric network comprising a) one or more three-dimensional binding sites on its surface, said binding sites being complementary in shape to at least a portion of the surface of a biomolecular target, and b) one or more high affinity molecules chemically anchored on the surface within said binding sites.

8. A hydrophilic nanoparticle comprising a crosslinked three-dimensional polymeric network comprising a) one or more three-dimensional binding sites on its surface, said binding sites being complementary in shape to at least a portion of the surface of a biomolecular target, and b) one or more high affinity molecules chemically anchored on the surface within said binding sites, said high affinity molecules being capable of a non-covalent binding interaction with a site on said target.

9. A nanoparticle according to claim 8 wherein said high affinity molecule is natural or synthetic and is selected from the group consisting of amino acids, peptides, polypeptides, proteins, glycoproteins, saccharides, polysaccharides, carbohydrates, lipopolysaccharides, nucleic acids, oligonucleic acids, porphyrins, substituted porphyrins, polyanions, polycations, organic compounds, steroids, steroid derivatives, drugs, enzymes, antibodies, antigens, cytokines, cellular receptors, cellular receptor fragments, and active agents.

10. A nanoparticle according to claim 8 having a diameter of from about 5 nm to about 400 nm.

11. A nanoparticle according to claim 8 having a diameter of from about 5 nm to about 200 nm.

12. A nanoparticle according to claim 8 having a diameter of from about 5 nm to about 100 nm.

13. A nanoparticle according to claim 7 wherein said high affinity molecule comprises amino acids.

14. A nanoparticle according to claim 7 wherein said high affinity molecule comprises nucleic acids.

15. A nanoparticle according to claim 7 wherein said high affinity molecule comprises saccharides.

16. A nanoparticle according to claim 7 wherein said scaffolding building blocks comprise carbohydrates.
